(19) United States
(12) Patent Application Publication
(10) Pub. No.: US 2024/0091016 A1
(43) Pub. Date: Mar. 21, 2024

(54) Rotatable Hip Prosthesis

(71) Applicant: Emory University, Atlanta, GA (US)

(72) Inventor: George Nelson Guild, III, Atlanta, GA (US)

(21) Appl. No.: 18/274,682

(22) PCT Filed: Jan. 27, 2022
(86) PCT No.: PCT/US2022/014119

§ 371 Date: Jul. 27, 2023

Related U.S. Application Data
(60) Provisional application No. 63/142,103, filed on Jan. 27, 2021.

(51) Int. Cl. A61F 2/36

(52) U.S. Cl. CPC A61F 2/3662; A61F 2/3609; A61F 2002/30329

(57) ABSTRACT

The disclosure relates to a femoral prosthesis device capable of preventing impingement and capable of being used with a constrained liner. In some examples, the device may include an elongated stem including a receiving section and a body section. The receiving section may have a bore. The device may include a calcar section including a through hole. The device may include an interface configured to be disposed on the receiving section between the body section and the proximal body. The device may include one or more fastener components configured to be disposed within the through hole and the bore when the stem and the proximal body are assembled so as to fasten/mate the elongated stem and the proximal body. When the stem and the proximal body are assembled and mated, the proximal body may be configured to radially rotate about the interface with respect to the elongated stem.

### CROSS-REFERENCE TO RELATED APPLICATIONS

This application claims the benefit of U.S. Provisional Application No. 63/142,103 filed Jan. 27, 2021. The entirety of this application is hereby incorporated by reference for all purposes.

### BACKGROUND

Peri-prosthetic hip dislocation is the number one cause for revision total hip arthroplasty. Worldwide, more than 1 million total hip replacements are performed each year (Ferguson et al, Hip Replacement, Lancet 2018, 392 (10158): 4662-1671), comprising a market size of nearly $7 billion in 2017. The rate of dislocation of first-time hip replacements varies between 0.2% and 10% annually, with some studies reporting an average dislocation rate of 1.9%. However, the rate of dislocation after revision or implant exchange surgery can be as high as 28% (Dargel et al., Dislocation Following Total Hip Replacement, Dtsch Arztebl Int 2014 December; 411(51-52):884-890). Of the six mechanisms for periprosthetic dislocation (acetabular malposition, femoral malposition, abductor insufficiency, impingement, late wear, and indeterminate) that have been identified, impingement has been the most common reasons for dislocation after hip replacement. Currently available prostheses have attempted to limit periprosthetic impingement by using larger femoral heads, dual mobility acetabular liners, constrained total hip liners, and optimized acetabular and femoral component positioning to prevent post-operative hip dislocation. However, the currently available prostheses, such as those that are used with a constrained hip liner, do not effectively avoid impingement over long-term.

### SUMMARY

Thus, there is a need for an improved prosthesis capable of preventing impingement and capable of being used with a constrained liner.

The devices of the disclosure may be a two piece femoral prosthesis in which a proximal body mates with a distal stem when assembled. The proximal body may be configured to rotate with respect to the stem when assembled (and implanted). This freedom of rotation of the proximal body that can remove impingement as a dislocation. The rotating proximal body can also allow for improved longevity if used with a constrained liner.

In some embodiments, the devices may include a (femoral) hip prosthesis device. The device may include an elongated stem including a receiving section and a body section. The receiving section may have a bore. The device may include a proximal body having a neck and a calcar section. The calcar section may include a through hole. The device may further include an interface configured to be disposed on the receiving section between the body section and the proximal body when the stem and the proximal body are assembled. The interface may include an opening. The device may further include one or more fastener components configured to be disposed within the through hole of the proximal body and the bore of the receiving section when the stem and the proximal body are assembled so as to fasten or mate the elongated stem and the proximal body. When the stem and the proximal body are assembled and mated, the proximal body may be configured to radially rotate about the interface with respect to the elongated stem.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 shows a view of an example of an assembled hip implant device according to some examples;

FIG. 2 shows an enlarged, partial view of FIG. 1;

FIG. 3 shows an exploded view of an example of a prototype of the device shown in FIG. 1;

FIG. 4 shows a view of another example of an assembled hip implant device according to some examples;

FIG. 5 shows an exploded view of the hip implant device shown in FIG. 4;

FIG. 6 shows another view of the hip implant device shown in FIG. 4;

FIG. 7 shows a cross-sectional view of the hip implant device shown in FIG. 6;

FIG. 8 shows an enlarged, partial view of the cross-sectional view of FIG. 7; and

FIG. 9 shows an exploded view of an example of a prototype of the device shown in FIG. 4.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

In some examples, the disclosure may relate to a hip implant device. The hip implant device may include a modular femoral prosthesis in which a proximal body may be mated with a distal stem when assembled. In some examples, the device may further include a fastener (e.g., screw) and an interface (e.g., polyethylene bushing) that are configured to rotatably mate the body with the stem when assembled and implanted.

When implanted, bony fixation may be obtained through fixation of the distal stem to bone. The proximal body may not be fixed to the bone, and the interface may be configured to allow rotation of the proximal body in either direction (anteversion or retroversion) with respect to the distal stem. In some examples, the interface may be configured to allow about 180° of rotation in either direction (anteversion or retroversion). By way of example, the interface may be configured to allow 360° of rotation of the proximal body with respect to the distal stem. This freedom of rotation of the proximal body can remove impingement as a dislocation mechanism from hip replacement, thereby overcoming the deficiencies of the currently available, static, femoral prosthesis. When implanted so that the neck of the proximal body contacts an acetabular cup (not shown), the femoral head (ball)(not shown) may be prevented from levering out because the proximal body is configured to rotate about its distal axis. This can allow a patient's leg position to continue moving without levering out the femoral head.

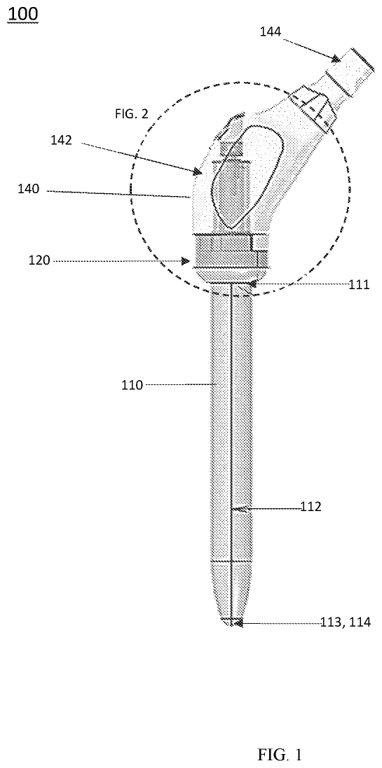

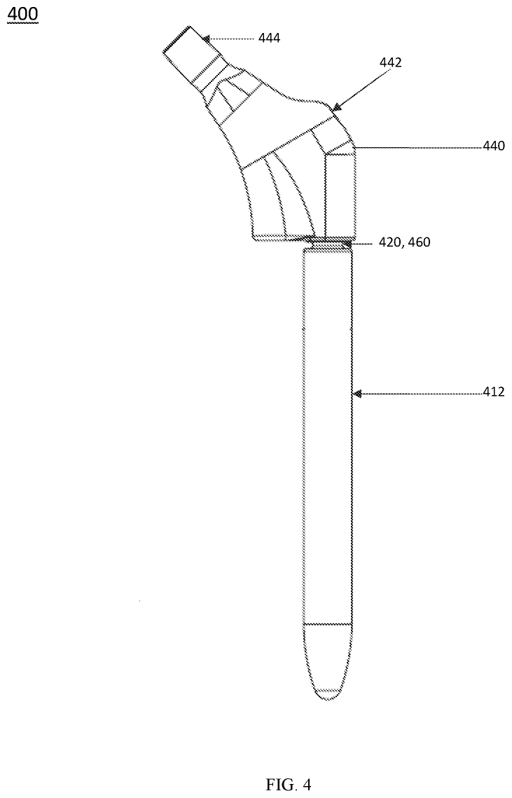

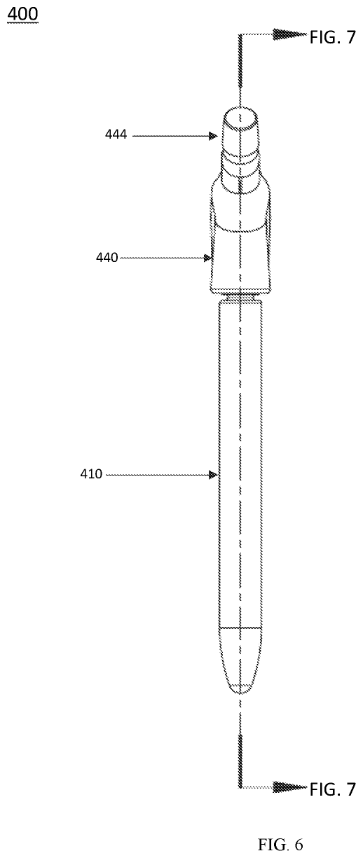

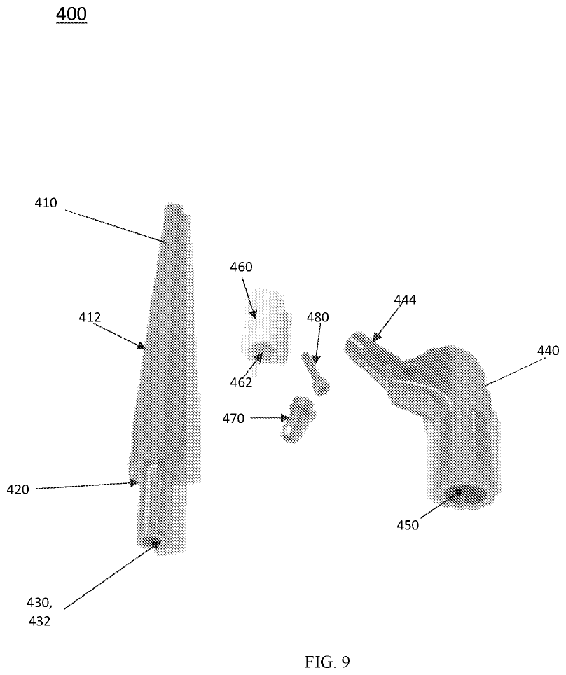

FIGS. 1-9 show examples of a hip implant (femoral prosthesis) device according to embodiments. FIGS. 1-3 show an example of a hip implant device 100 according to some embodiments and FIGS. 4-9 show an example of a hip implant device 400 according to some embodiments. In some examples, the device 100 may be configured for a femur replacement procedure for a patient with extensive proximal femoral bone loss and the device 400 may be configured for krevision procedure for a patient with moderate proximal femoral bone loss. For example, the component(s) (e.g., proximal body) of the device 100 may be larger (e.g., larger diameter) than the respective component(s) of the device 400 so as to accommodate more extensive proximal femoral bone loss.

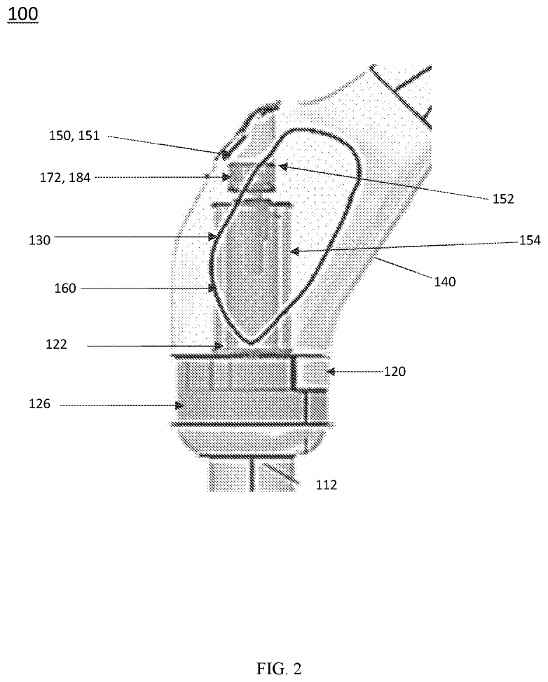

As shown in FIGS. 1-3, in some examples, the device 100 may include a stem 110, a proximal body 140, and an interface 160 disposed therebetween when assembled. The body 140 may be configured to rotate about the interface 160 with respect to the stem 110 (when assembled and fastened). It will be understood that a portion of the device 100 that is shown transparent in FIGS. 1 and 2 is for illustrative purpose only so that the innerworkings of the device 100 could be visualized for reference.

In some examples, the stem 110 may include a body section 112 and a receiving section 120. The body section 112 may include a first end 111, a second 113, and a length therebetween. The receiving section 120 may be disposed to extend from the first end 111 and configured to mate with the proximal body 140 when assembled. In examples, the stem 110 may include a distal tip 114 at the second end 113. The body section 112 may be configured to be implanted within a femoral bone.

In some examples, the receiving section 120 may be configured to receive one or more fastener components (e.g., 170 and 180) that can rotatably fasten or mate the proximal body 140 to the stem 110 when assembled. In some examples, the receiving section 120 may include one or more portions complimentary to one or more fastener components. In some embodiments, the receiving section 120 may include a first portion 122, a second portion 124, and a third portion 126. In some examples, the portions may have a different diameter. For example, as shown in FIGS. 1-3, the first portion 122 may have an outer diameter that is smaller than an outer diameter of the body section 112 of the stem 110, the second portion 124, and the third portion 126. The outer diameter of the second portion 124 may be larger than the first portion 122 and smaller than the outer diameter of the third portion 126. The outer diameter of the third portion 126 may be larger the outer diameters of the first portion 122, the second portion 124, and the body section 112.

In some examples, the receiving section 120 may include a bore 130 disposed along the length of the receiving section 120. The bore 130 may have a first end (opening 132) in the first portion 122, a second end (not shown) disposed in the third portion 126, and a length therebetween (through the second portion 124). In some embodiments, the bore 130 may have threads 134 disposed on its inner surface along its length complimentary to the one or more fastener components (e.g., bolt 180). In some embodiments, the shape of the bore opening 132 (and along of the portion of the length) may correspond to an outer diameter of at least a portion of one of the fastener members (e.g., mounting member 170). In some examples, the receiving section 120 may be a trunnion, such as a male trunnion.

In some embodiments, the receiving section 120 may include a notch 123 that is disposed on an outer surface of the second portion 124 along a portion of its outer circumference on an upper surface of the third portion 126. The notch 123 may be configured to restrict the rotation of the proximal body 140 when assembled and mated with the stem 110. In this example, the notch 123 may be configured to restrict rotation of the proximal body 140 to about 180 degrees with respect to the stem 110. In some examples, the notch 123 may be configured to restrict the rotation to more or less than 180 degrees, for example, by changing the circumference size of the notch 123 with respect to the portions of the receiving section 120. In other examples, the notch 123 may be omitted.

In some embodiments, the proximal body 140 may include a calcar section 142 and a neck portion 144 configured to connect to a head component (not shown). The calcar section 142 may include a through hole 150 configured to receive the one or more fastener components (e.g., 170 and 180) and the interface 160.

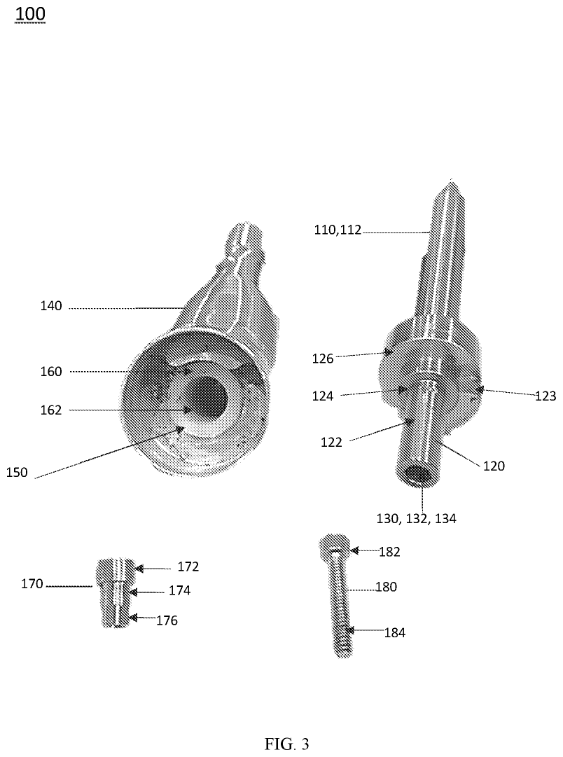

In some examples, the through hole 150 may include one or more sections configured to receive the interface 160 and/or one or more of the fastener components (170 and/or 180). For example, as shown in FIGS. 1 and 2, the through hole 150 may include a first section 152 and a second section 154. The diameter of the second section 154 may be larger than the diameter of first section 152. In some examples, the interface 160 may be configured to be disposed within the second section 154, as shown in FIG. 3.

In some examples, when the device 100 is assembled, the interface 160 may be configured to be disposed between the stem 110 and the proximal body 140, and to allow rotation of the proximal body 140 with respect to the stem 110 while receiving the force from that rotatable movement to minimize wear of the stem 110 and/or the proximal body 140 resulting from the rotatable movement. As shown in FIGS. 1-3, when the device 100 is assembled, the interface 160 may be configured to be disposed between the first portion 122 and the second section 154 so as to surround the outer surface of the first portion 122 and within the second section 154 of the proximal body 140.

In some embodiments, the interface 160 may be a hollow component. In some examples, the interface 160 may be a polyethylene bushing. In other examples, the interface 160 may be made of additional and/or alternative materials (e.g., other plastic material(s), polymer(s), etc.). In some examples, the interface 160 may have smooth inner and/or outer surface(s). In some examples, the interface 160 may have textured inner and/or outer surface(s).

In some examples, a length of the interface 160 may correspond to a length of the first portion 122 of the receiving section 120 so that the interface 160 is flush with the second portion 124 when the device 100 is assembled. In some examples, a length of the interface 160 may be longer than a length of the second section 154 of the proximal body 140 so as to provide an exposed area of the interface 160 (on the first portion 122 of the receiving section 120). In some examples, when the device 100 is assembled, the interface 160 may include an opening 162 to its inner channel corresponding to an opening 151/diameter of the section 152 of the through hole 150.

In some examples, the one or more fastener components may include a bolt (or screw) 180 and a mounting member (e.g., boss) 170 having a through hole through which the bolt 180 can be disposed. In some examples, the screw/bolt 180 may include a head 182 and a shaft 184. The head 182 may be any head, including but not limited to flat, slotted, external hex, among others, or any combination thereof. The shaft 184 may include threads complimentary to and configured to engage the threaded inner surface 134 of the bore 130.

In some examples, the mounting member 170 may include one or more sections. For example, the mounting member 170 may include a first section 172, a second section 174, and a third section 176 as shown in FIG. 3. In some examples, the first section 172 may have an outer diameter larger than outer diameters of the second section 174 and the third section 176. In some examples, the third section 176 may have a different outer shape than the outer shapes of the first section 172 and the second section 174. For example, the third section 176 may have a shape complimentary to the shape of the bore 130 disposed within a portion. By way of example, the third section 176 may have a square outer shape that corresponds to a square shape of the bore 130 within the third portion 126. In this example, the first section 172 and the second section 174 may have a round or circular outer shape.

In some examples, the mounting member 170 may have a length that is shorter than a length of the bolt 180. As shown in FIGS. 1 and 2, the shaft 184 may be configured to extend past the end of the mounting member 170 when the bolt 180 is disposed in the mounting member 170.

In some examples, to assembly the device 100, the interface 160 may be disposed between the stem 110 and the proximal body 140 so that the interface 160 is disposed along and between the second section 154 and the first portion 122. In some examples, the interface 160 may have an outer diameter that is the substantially the same as the outer diameter of the second portion 124 so that it is flushed with the second portion 124 when assembled to surround the first portion 122. In some examples, a portion of the interface 160 may be exposed between the body portion 112 and the proximal body 140 when the proximal body 140 and the stem 110 are assembled. The screw/bolt 180 may be assembled with the mounting member 170 so that the head 182 may be disposed on/within the first section 172. In some examples, the assembled fastening components (screw 180/mounting member 170) may then be disposed in the opening 151 into the through hole 150/the bore 130 so that the first section 172 engages the first section 152/the bore 130 of the first portion 122, the second section 174 engages the bore 130 of the first portion 122/the second portion 124, and the third section 176 engages with a complimentary portion of the bore 130 within the third portion 126. This way, when the fastening components (170 and 180) are fastened in the assembled device 100, the fastening components (170 and 180) can be configured to prevent a pistoning movement while allowing rotation of the proximal body 140 about the interface 160 with respect the stem 110.

FIGS. 4-9 show an example of a hip implant device, for example, for a patient with moderate proximal femoral bone loss according to some embodiments. In some examples, as shown in FIGS. 4-9, the device 400 may include a stem 410, a proximal body 440, and an interface 460 disposed therebetween when assembled. The body 440 may be configured to rotate about the interface 460 with respect to the stem 410 (when the device 400 is assembled and fastened).

In some examples, the stem 410 may include a body section 412 and a receiving section 420. The body section 412 may include a first end 411, a second 413, and a length therebetween. The receiving section 420 may be disposed to extend from the first end 411 and configured to mate with the proximal body 440. In examples, the stem 410 may include a distal tip 414 at the second end 413. The body section 412 may be configured to be implanted within a femoral bone.

In some examples, the receiving section 420 may be configured to receive one or more fastener components (e.g., 470 and 480) that can rotatably fasten or mate the proximal body 440 to the stem 410 when assembled. In some examples, the receiving section 420 may include one or more portions that is complimentary to the one or more fastener components (e.g., 470 and 480). In some examples, the receiving member 420 may include a first portion 422 and a second portion 424 that may have different dimensions and/or shapes of their respective inner and/or outer diameters. In some examples, the first portion 422 and second portion 424 may have an outer diameter that is smaller than an outer diameter of the body section 412 of the stem 410. In some examples, the outer diameters of the first portion 422 and the second portion 424 may be the same, and the inner diameter of the bore 430 of the first portion 422 and the second portion 424 may be different.

In some examples, the receiving section 420 may include a bore 430 disposed along the length of the receiving section 420. The bore 430 may have a first end (opening 432) in the first portion 422 and a second end 434 disposed in the second portion 424, and a length therebetween. In some embodiments, the bore 430 may have threads 434 disposed on its inner surface along its length complimentary to the one or more fastener components (e.g., bolt 480). In some embodiments, the shape of the bore opening 432 (and along of the portion of the length) may correspond to an outer diameter of at least a portion of one of the fastener members (e.g., mounting member 470).

In some embodiments, the proximal body 440 may include a calcar section 442 and a neck portion 444 configured to connect to a head component (not shown). The calcar section 442 may include a through hole 450 configured to receive the one or more fastener components and the interface 460.

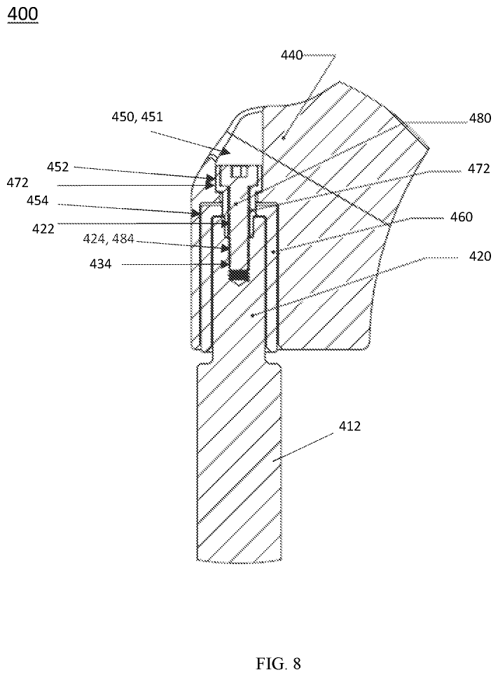

In some examples, the through hole 450 may include one or more sections configured to receive the interface 460 and/or one or more of the fastener components (470 and/or 480). For example, as shown in FIG. 8, the through hole 450 may include first section 452 and a second section 454. The diameter of the second section 454 may be larger than the diameter of first section 452. When the proximal body 440 is disposed on/mated with the receiving section 420 of the stem 410, the second section 454 may be configured to surround the outer surface of the receiving member 420 with the interface 460 disposed therebetween. As shown, the interface 460 may be disposed along its length and on the outer surface of the receiving section 420.

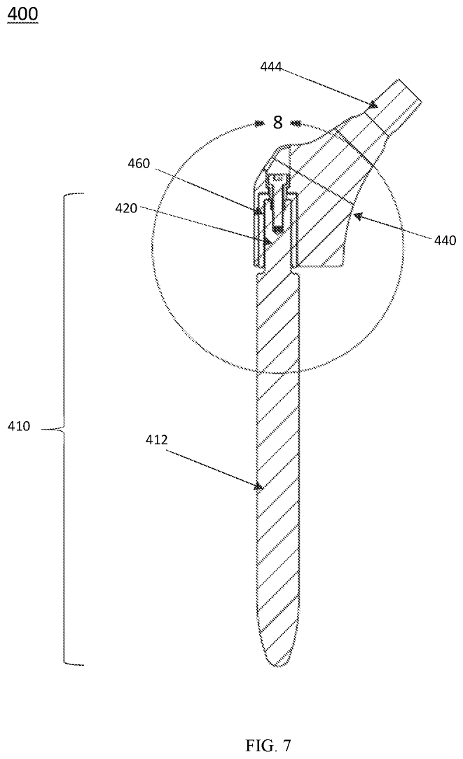

In some examples, like the device 100/interface 160, when the device 400 is assembled, the interface 460 may be configured to be disposed between the stem 410 and the proximal body 440, and to allow rotation of the proximal body 440 with respect to the stem 410 while receiving the force from that rotatable movement to minimize wear of the stem 410 and/or the proximal body 440 resulting from the rotatable movement. As shown in FIGS. 7 and 8, when the device 400 is assembled, the interface 460 may be configured to be disposed between the receiving section 420 and the second section 454 so as to surround the outer surface of receiving section 420 and within the second section 454 of the proximal body 440.

In some embodiments, the interface 460 may be a hollow component. In some examples, the interface 460 may be a polyethylene bushing. In other examples, the interface 460 may be made of additional and/or alternative materials (e.g., other plastic material(s), polymer(s), etc.). In some examples, the interface 460 may have smooth inner and/or outer surface(s). In some examples, the interface 460 may have textured inner and/or outer surface(s).

In some examples, a length of the interface 460 may correspond to a length of the receiving section 420 when the device 400 is assembled. In some examples, a length of the interface 460 may be longer than a length of the second section 454 of the proximal body 440 so as to provide an exposed area of the interface 460 (on the receiving section 420). In some examples, when the device 400 is assembled, the interface 460 may include an opening 462 to its inner channel corresponding to an opening 451/diameter of the section 452 of the through hole 450.

In some examples, the one or more fastener components may include a bolt (or screw) 480 and a mounting member (e.g., boss) 470 having a through hole through which the bolt 480 can be disposed. In some examples, the bolt 480 may include a head 482 and a shaft 484. The head 482 may be any head, including but not limited to flat, slotted, external hex, among others, or any combination thereof. The shaft 484 may include threads complimentary to and configured to engage the threaded inner surface 434 of the bore/receiving member 430.

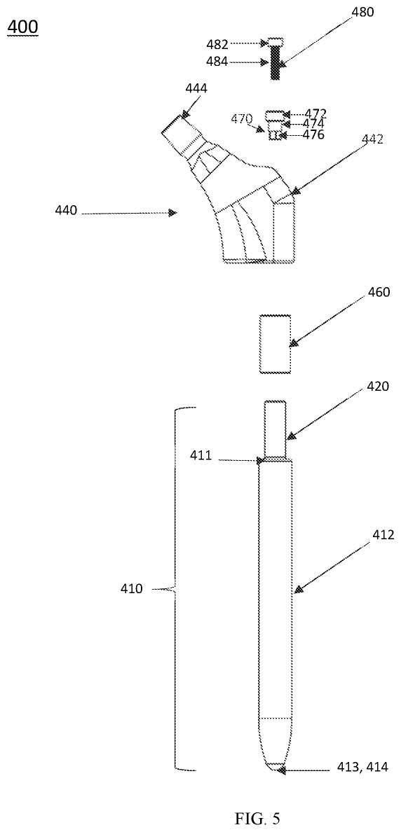

In some examples, the mounting member 470 may include one or more sections. For example, the mounting member 470 may include a first section 472, a second section 474, and a third section 476 as shown in FIG. 5. The first section 472 may have an outer diameter larger than outer diameters of the second section 474 and third section 476. In some examples, the third section 476 may have a different outer shape than the outer shapes of the first section 472 and the second section 474. For example, the third section 476 may have a shape complimentary to the shape of the bore 430 within at least a portion of its length. For example, the third section 476 may have a square outer shape that corresponds to a square shape within the bore 430. In this example, the first section 472 and the second section 474 may have a round or circular outer shape.

In some examples, the bolt 470 may have a length that is shorter than a length of the bolt 480. As shown in FIGS. 7 and 8, the shaft 484 may be configured to extend past the end of the mounting member 470 when the bolt 480 is disposed in the mounting member 470.

In some examples, to assembly the device 400, the interface 460 may be disposed between the stem 410 and the proximal body 440 so that the interface 460 is disposed along the second section 454 and the first portion 422. In some examples, a portion of the interface 460 may be exposed between the body portion 412 and the proximal body 440. The screw/bolt 480 may be assembled with the mounting member 470 so that the head 482 may be disposed on/within the first section 472. In some examples, the assembled fastening components (screw 480/mounting member 470) may then be disposed in the opening 451 into the through hole 450/bore 430 so that the first section 472 engages with the first section 452, the second section 474 engages with the bore 430 of the first portion 422, and the third section 176 engages with a complimentary portion of the bore 430 within the second portion 424. This way, when the fastening components (470 and 480) are fastened in the assembled device 400, the fastening components (470 and 480) can be configured to prevent a pistoning movement while allowing rotation of the proximal body 440 about the interface 460 with respect the stem 410.

In some examples, the stem 110/410 and the proximal body 140/440 may be made of any known biocompatible implant material(s), including but not limited to one or more metals (e.g., titanium alloys, stainless steel, special high-strength alloys, alumina, zirconia, zirconia toughened alumina (ZTA), and UHMWPE, etc.), among others, or a combination thereof. In some examples, as shown in the figures, the stem 110/410 and the proximal body 140/440 may have smooth outer surfaces. In some examples, the stem 110/410 and/or the proximal body 140/440 may include one or more outer surfaces that are textured, coated (e.g., with polymer), among others, or any combination thereof.

In some examples, the devices 100 and/or 400 may be used in revision surgery of the hip, for example, to replace an existing hip implant. The device used may depend on the amount of proximate femoral bone loss. By way of example, in revision surgery using the devices 100 or 400, the hip may be exposed by the surgeon through any method of exposure, such as posterior approach, lateral approach, and/or anterior based approach. Then subcutaneous dissection and fascial incision with exposure of the hip may be obtained so that the total hip prosthesis in place can be examined by the surgeon. For example, the existing prosthetic hip can be dislocated and the proximal femur and existing stem cab be examined. The head ball can also be disengaged from the trunnion using a bone tamp and inspected for corrosion. If the existing stem is well fixed to bone than appropriate removal techniques with the appropriate instrument(s), such as high-speed burs, size, and/or osteotomes, can be utilized to remove the existing hip stem minimizing damage to the femoral bone. After removal and examination, the diaphyseal bone of the femur may be increased in size so that the sore auricle bone is reached. The surgeon may then insert trial components of the device 100/400 into the exposed bone to determine the correct size of the stem 110/410, proximal body 140/440, and head ball (not shown). The surgeon may use the trial components to ensure that sufficient amount of proximal femoral bone is removed, for example, using a rongeur, to allow adequate rotation of the proximal femoral body thereby removing impingement that can cause dislocation. Additionally, a range of motion testing flexion internal rotation, as well as extension external rotation, may be performed to ensure that the hip is stable throughout a range of motion with trial components. Once that has been confirmed, the surgeon can dislocate the hip with a bone hook, remove the trial femoral head, unscrew the locking bolt of the proximal femoral body so that the proximal body can be removed off of the distal stem. Once the size of the desired proximal body and stem has been selected, the device 100/400 may be implanted.

For example, with regards to device 100, as shown in FIGS. 1 and 2, after the stem 110 is inserted into the femoral bone, the interface 160 may be positioned onto the receiving section 120 of the stem 110. In some examples, the interface 160 may be disposed (i) within the second section 154 of the proximal body 140 or (ii) on the first section 122 of the receiving section 120. The proximal body 140 may be mated to the stem 110 so that the second section 154 and the interface 160 surround the first portion 122. After the proximal body 140 is placed onto the first portion 122 so that the interface 160 surrounds the receiving portion 122 and its through hole 150/opening 151 may align with the opening 132 of the bore 130 of the receiving section 120 of the stem 110. After which, the bolt 180 may be inserted into the mounting member 170. The assembled mounting member 170 and the bolt 180 may then be positioned into the channel defined by the aligned through hole 150/bore 130/openings 132, 151 so that the first section 172 is disposed on/within the first portion 122 and the third section 176 is disposed at the complimentary portion within the bore 130 of the third portion 126. The bolt 180 may be screwed using its respective instrument via the head 182 so that its threads 184 engage the complimentary inner surface threads 134 of the bore 130 resulting in the proximal body 140 and the stem 110 being mated.

After the mounting member 170/bolt 180 is fastened, the interface 160 can enable the proximal body 130 to radially rotate (e.g., anteversion or retroversion) about 180 degrees about the interface 160 with respect to the stem 110 due to the notch 123. In some examples, the notch 123 may be modified and/or omitted so that the proximal body 140 may be configured to rotate more or less degrees (for example, about 360 degrees) about the interface 160 with respect to the stem 110 when the screw 180 is fastened/mated. When implanted, the proximal body 140 may be configurated to radially rotate about the interface 160 with respect to the stem 110 and therefore remove any impingement while focusing wear from that rotation at the interface 160.

With regards to device 400, as shown in FIGS. 7 and 8, after the stem 410 is inserted into the femoral bone, the proximal body 440 may be mated to the stem 410 by placing the interface 460 onto the receiving member 420. After the interface 460 is placed onto the receiving member 420, the proximal body 440 may be placed onto the interface 460 so that its through hole 450/opening 451 aligns with the opening 432 of the bore 430 of the receiving section 420 of the stem 410. After which, the bolt 480 may be inserted into the mounting member 470. The assembled mounting member 470 and the bolt 480 may be positioned in the channel defined by the aligned through hole 450/bore 150/openings 451, 432. The bolt 480 may be screwed using its respective instrument via the head 482 so that its threads 484 engage the complimentary inner surface threads 434 of the bore 430 of the receiving section 420 resulting in the proximal body 440 and the stem 410 being mated.

Like the device 100, after the screw/mounting member 470/480 is fastened, the interface 460 can enable the proximal body 470 to radially rotate (e.g., anteversion or retroversion) with respect to the stem 410. In this example, the proximal body 440 may be configured to rotate about 180 degrees in either direction about the interface 460 with respect to the stem 410 when the device 400 is assembled and fastened. In some examples, the proximal body 440 may be configured to rotate in 360 degrees about the interface 460 with respect to the stem 410 when the device 400 is assembled and fastened. When implanted, like the device 100, the proximal body 440 may be configurated to radially rotate about the interface 460 with respect to the stem 410 and therefore remove any impingement while focusing wear from that rotation at the interface 460.

As will be described herein, the devices 100 and 400 may include modular components that may be selectively attached to each other during a surgical procedure. In this regard, each of the stem 110/410, the proximal femoral body 140/440, and/or the femoral head (not shown)(if included) can include a plurality of unique components having various dimensions that may be suitable for a particular patient. As such, a surgeon may have a kit of components including various distal stems, proximal femoral bodies and femoral heads all having various dimensions and features that the surgeon can intraoperatively select according to a patient's particular need.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed is:

1. A hip prosthesis, comprising:
an elongated stem including a receiving section and a body section, the receiving section having a bore;
a proximal body having a neck and a calcar section;
the calcar section including a through hole;
an interface configured to be disposed on the receiving section between the body section and the proximal body when the stem and the proximal body are assembled, the interface including an opening;
one or more fastener components configured to be disposed within the through hole of the proximal body and the bore of the receiving section when the stem and the proximal body are assembled so as to fasten or mate the elongated stem and the proximal body; and
wherein when the stem and the proximal body are assembled and mated, the proximal body is configured to radially rotate about the interface with respect to the elongated stem.

2. The hip prosthesis according to claim 1, wherein the interface is a polyethylene bushing.

3. The hip prosthesis according to claim 2, wherein a length of interface is longer than a length of the through hole.

4. The hip prosthesis according to claim 2, wherein when the stem and proximal body are mated and fastened, a portion of the interface between the proximal body and the body section is exposed.

5. The hip prosthesis according to claim 3, wherein the one or more fastener components includes a bolt, the bolt including a head and a shaft.

6. The hip prosthesis according to claim 5, wherein the one or more fastener components includes a mounting member having a through hole through which a bolt is configured to be disposed when assembled.

7. The hip prosthesis according to claim 6, wherein the mounting member is a boss.

8. The hip prosthesis according to claim 6, wherein:
the mounting member includes one or more sections;
the one or more section includes a first section, a second section, and a third section;
a first section has an outer diameter that is larger than an outer diameter of the second section; and
the third section has an outer diameter having a shape that is different from a shape of the outer diameter of the first section and a shape of the outer diameter of the second section.

9. The hip prosthesis according to claim 8, wherein the mounting member has a length that is shorter than a length of a shaft of the bolt so that the shaft of the bolt extends past the mounting member when disposed in the mounting member.

10. The hip prosthesis according to claim 6, wherein:
the through hole of the proximal body includes one or more sections, the one or more sections including a first section and a second section; and
when the proximal body and the stem are assembled, the second section is configured to be disposed so as to surround the interface and the receiving section.

11. The hip prosthesis according to claim 10, wherein:
a diameter of the second section is larger than a diameter of the first section.

12. The hip prosthesis according to claim 10, wherein:
the receiving section includes one or more portions, the receiving section including a first portion, a second portion, and a third portion;
the first portion has an outer diameter that is smaller than an outer diameter of the second portion and an outer diameter of the third portion; and
the outer diameter of the second portion is smaller than the outer diameter of the third portion.

13. The hip prosthesis according to claim 12, wherein when the proximal body and the stem are assembled, the interface is disposed to surround the first portion of the receiving section.

14. The hip prosthesis according to claim 12, wherein:
the proximal body includes a notch disposed on the third portion and disposed to surround at least a part of an outer circumference of the second portion; and
the notch is configured to restrict an amount of rotation of the proximal body about the interface with respect to the stem when the proximal body and the stem are assembled and fastened.

15. The hip prosthesis according to claim 14, wherein:
when the proximal body and the stem are assembled, the interface is disposed to (i) surround the first portion of the receiving portion and (ii) above and flush with the second portion of the receiving section.

16. The hip prosthesis according to claim 13, wherein the mounting member includes a section having a different outer shape and the bore of the receiving section having a portion with a diameter corresponding to the outer shape.

17. The hip prosthesis according to claim 16, wherein the third section of the mounting member has the different outer shape.
